(19) United States
(12) Patent Application Publication
(10) Pub. No.: US 2006/0052640 A1
(43) Pub. Date: Mar. 9, 2006

(54) Method for the production of substituted trifluroethylenses

(76) Inventors: Peter Wolfrum, Monheim (DE); Albrecht Marhold, Leverkusen (DE)

Correspondence Address: BAYER CROPSCIENCE LP;Patent Department, 100 BAYER ROAD, PITTSBURGH, PA, 15205-9741, US

(21) Appl. No.: 10/514,266

(22) PCT Filed: May 8, 2003
(86) PCT No.: PCT/EP03/04810

§ 371 Date: Jul. 21, 2005

(30) Foreign Application Priority Data
May 13, 2002 (DE) 102 21 119.1

(51) Int. Cl. C07C 17/25

(52) U.S. Cl. 564/209; 570/155

(57) ABSTRACT

The present invention relates to a method for the production of substituted trifluoroethylenes, in which a compound of the formula (II) 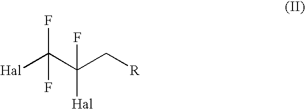 in which
R is C₁-C₆-alkyl optionally substituted by carboxyl, halogen, C₁-C₆-alkoxy, C₁-C₆-alkoxycarbonyl or C₁-C₄-alkylcarbonyloxy or is the grouping 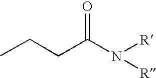
   in which R′ and R″ are, independently of one another, hydrogen or C₁-C₆-alkyl or the two, together with the nitrogen atom to which they are bonded, are saturated cycloalkyl with 3 to 5 carbon atoms, in which a ring member is optionally replaced by oxygen or sulfur and which is optionally substituted by one to four C₁-C₄-alkyl, trifluoromethyl or C₁-C₄-alkoxy, and Hal is chlorine, bromine or iodine, is brought into contact with a dehalogenating agent in a polyol or its ethers.

The present invention relates to a method for the production of substituted trifluoroethylenes.

Trifluoroethylenes have been known for some time. Thus, trifluoroethylenes, such as, e.g., 1,1,2-trifluoro-4-bromo-1-butene, are important intermediates in the production of agrochemicals, in particular compounds having an insecticidal and nematicidal effect (cf. U.S. Pat. No. 3,513,172, WO 86/07590, EP 342 150 A1, EP 507 464 A1, WO 02/06260, WO 02/06256, WO 02/06257 and WO 02/06259).

Trifluoroethylenes can be reduced, e.g., by dehalogenation of trifluorodihaloethane derivatives. Thus, Tarrant et al. (J. Org. Chem., 34 (2), 323 (1969)) describe the dehalogenation of 1,4-dibromo-2-chloro-1,1,2-trifluorobutane using zinc in ethanol. EP 334 796 A1 discloses the dehalogenation of the same starting material electro-chemically in methanol. A common feature of both methods is that the reaction is carried out in a short-chain alcohol. However, the methods known hitherto for the production of these intermediates exhibit a number of disadvantages. Thus, the yields, e.g., are relatively low. It was therefore an object of the present invention to make available a method which makes possible the production of trifluoroethenes with good yields and with simple purification.

A method for the production of substituted trifluoroethylenes of the formula (I) has now been found, 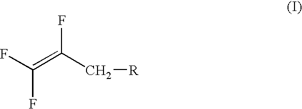
in which
R is C₁-C₆-alkyl optionally substituted by carboxyl, halogen, C₁-C₆-alkoxy, C₁-C₆-alkoxycarbonyl or C₁-C₄-alkylcarbonyloxy or is the grouping 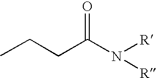
   in which
   R′ and R″ are, independently of one another, hydrogen or C₁-C₆-alkyl or the two, together with the nitrogen atom to which they are bonded, are saturated cycloalkyl with 3 to 5 carbon atoms, in which a ring member is optionally replaced by oxygen or sulfur and which is optionally substituted by one to four C₁-C₄-alkyl, trifluoromethyl or C₁-C₄-alkoxy,
      characterized in that a compound of the formula (II) 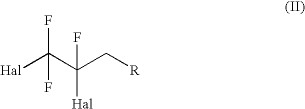
in which
R has the abovementioned meaning, and
Hal is chlorine, bromine or iodine, preferably chlorine or bromine, is brought into contact with a dehalogenating agent in a polyol or its ethers.

Formula (I) also includes the metal salts, in particular of the carboxylic acids according to the invention.

Particularly preferred definitions of the compounds of the formula (I) which are produced using the method according to the invention are listed below.
R is preferably methyl, ethyl, n-propyl, isopropyl, n-butyl, isobutyl, s-butyl or t-butyl, in each case optionally substituted by carboxyl, fluorine, chlorine, bromine, methoxy, ethoxy, n-propoxy, isopropoxy, n-butoxy, isobutoxy, s-butoxy, t-butoxy, n-pentoxy, isopentoxy, methoxycarbonyl, ethoxycarbonyl, n-propoxycarbonyl, isopropoxycarbonyl, n-butoxycarbonyl, isobutoxy-carbonyl, s-butoxycarbonyl, t-butoxycarbonyl, methylcarbonyloxy, ethyl-carbonyloxy, n-propylcarbonyloxy, isopropylcarbonyloxy, n-butylcarbonyloxy, isobutylcarbonyloxy, s-butylcarbonyloxy or t-butylcarbonyloxy.
R is furthermore preferably the grouping 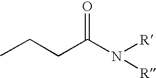
   in which
   R′ and R″ are, independently of one another, hydrogen, methyl, ethyl, n-propyl, isopropyl, n-butyl, isobutyl, s-butyl or t-butyl or the two, together with the nitrogen atom to which they are bonded, are one of the following rings: 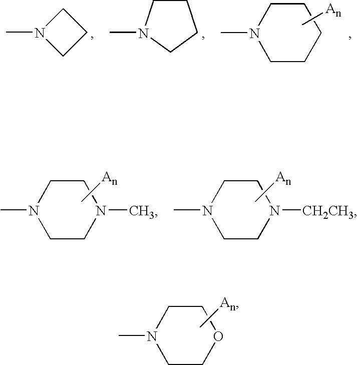
   in which
   A is methyl, ethyl, n-propyl, isopropyl or s-propyl, and
   n is 0, 1, 2, 3 or 4.
R is particularly preferably methyl or ethyl, in each case optionally substituted by carboxyl, fluorine, chlorine, bromine, methoxy, ethoxy, n-propoxy, isopropoxy, n-butoxy, isobutoxy, s-butoxy, t-butoxy, n-pentoxy, isopentoxy, methoxycarbonyl, ethoxycarbonyl, n-propoxycarbonyl, isopropoxycarbonyl, n-butoxycarbonyl, isobutoxycarbonyl, s-butoxycarbonyl, t-butoxycarbonyl, methylcarbonyloxy, ethylcarbonyloxy, n-propylcarbonyloxy, isopropyl-carbonyloxy, n-butylcarbonyloxy, isobutylcarbonyloxy, s-butylcarbonyloxy or t-butylcarbonyloxy.
R is furthermore particularly preferably the grouping 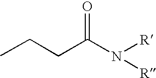
   in which

R′ and R″ are, independently of one another, hydrogen, methyl or ethyl or the two, together with the nitrogen atom to which they are bonded, are one of the following rings:

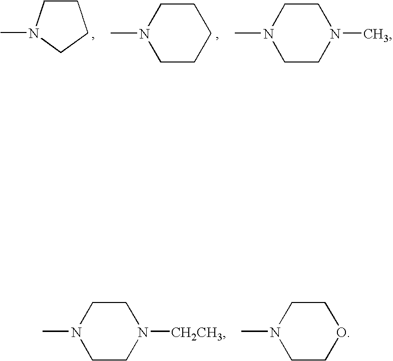

The term “polyols” as used herein refers to alcohols with more than one OH group, preferably to alcohols with 2 or 3 OH groups.

Examples of suitable polyols are ethylene glycol, propylene glycol, diethylene glycol, N-methyldiethanolamine, triethanolamine or glycerol, and their ethers, for example ethylene glycol monoethyl ether, ethylene glycol monobutyl ether, propylene glycol monomethyl ether, propylene glycol monoethyl ether, propylene glycol monobutyl ether, diethylene glycol monomethyl ether or diethylene glycol monoethyl ether.

Particularly preferred polyols or ethers of polyols used in the method according to the invention are diethylene glycol, diethylene glycol monomethyl ether, N-methyldiethanolamine or triethanolamine.

Very particularly preferably, triethanolamine is used in the method according to the invention.

Suitable dehalogenating agents include, e.g., metals, such as iron, zinc, magnesium, aluminum, tin, copper or nickel. Iron and/or zinc are particularly suitable for use in the method according to the invention. Mixtures of the metals or forms activated by additions of metals, such as, e.g., metal salts, can also be used according to the invention.

The reaction can be carried out within a broad temperature range. It is preferably carried out at temperatures of 0 to 100° C., temperatures of 20 to 65° C. being preferred.

In a preferred embodiment of the method, the isolation of the compounds of the formula (I) is carried out directly by distillation from the reaction mixture. Other routes for the isolation can also be used; however, the distillation makes possible an immediate and efficient separation of the product from the high-boiling solvent. Furthermore, the remaining solvent can be reused after filtering off the metals/metal salts.

The starting compounds of the formula (II) can be produced according to methods known per se, e.g. analogously to U.S. Pat. No. 3,562,341; Chin. J. Chem., 281 (1990); J. Org. Chem., 29, 1198 (1964); J. Fluorine Chem., 66, 171 (1994); Tetrahedron Lett., 31, 1307 (1990).

The following examples illustrate the method according to the invention. The method according to the invention can also, in an analogous way, be carried out with other abovementioned starting compounds, polyols or ethers thereof and with other dehalogenating agents. The examples are therefore not to be interpreted in a limiting fashion.

### EXAMPLE 1

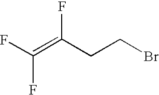

131 g (2 mol) of zinc dust are introduced into 1000 ml of diethylene glycol in an apparatus equipped with a stirrer and are suspended by stirring. After warming to 50° C., 500 g (1.64 mol) of 1,4-dibromo-2-chloro-1,1,2-trifluorobutane are metered in inside 2 hours. A slightly exothermic reaction arises and the metering rate is adjusted so that the internal temperature is maintained at 50-55° C. without additional external warming. After the reactant has finished being metered in, stirring is carried out for a further 30 minutes, then vacuum is applied and the product is distilled. The bulk of the product distills over at a boiling point of 40-50° C./200 mbar. The yield is 302 g and the content is 92%, which corresponds to a yield of 89% of theory.

### EXAMPLE 2

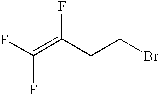

104 g (1.59 mol) of zinc, 1 g of ZnCl₂ and 500 ml of diethylene glycol monomethyl ether are introduced into an apparatus equipped with a stirrer. 250 g (0.82 mol) of 1,4-dibromo-2-chloro-1,1,2-trifluorobutane are added dropwise at an internal temperature of 45-50° C. After the reaction temperature has risen to 50° C., the amount metered in is adjusted so that the reaction takes place at 50 to 55° C. After the end of the addition, the mixture is stirred for an additional 40 minutes and the pressure is subsequently reduced until the product distills off via a bridge-type stillhead. The yield is 144 g and the content, by GC analysis, is 93.2%.

1-5. (canceled)

6. A method for the production of compounds of formula (I) 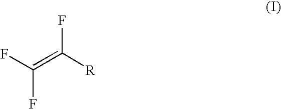
in which
R is C₁-C₆-alkyl optionally substituted by carboxyl, halogen, C₁-C₆-alkoxy, C₁-C₆-alkoxycarbonyl, or C₁-C₄-alkylcarbonyloxy or is the group 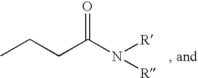
and
R′ and R″ are, independently of one another, hydrogen or C₁-C₆-alkyl, or R′ and R″ together with the nitrogen atom to which they are bonded are saturated cycloalkyl with 3 to 5 carbon atoms in which a ring member is optionally replaced by oxygen or sulfur and that is optionally substituted by one to four C₁-C₄-alkyl, trifluoromethyl, or C₁-C₄-alkoxy,
comprising contacting a compound of formula (II) 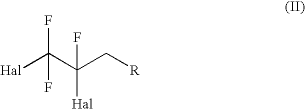
in which
R has the has the meaning given for formula (I), and
Hal is chlorine, bromine, or iodine,
with a dehalogenating agent in a polyol or an ether of a polyol.

7. The method according to claim 6 in which the polyol or ether of a polyol is ethylene glycol, propylene glycol, diethylene glycol, N-methyldiethanol-amine, triethanolamine, glycerol, ethylene glycol monoethyl ether, ethylene glycol monobutyl ether, propylene glycol monomethyl ether, propylene glycol monoethyl ether, propylene glycol monobutyl ether, diethylene glycol monomethyl ether, or diethylene glycol monoethyl ether.

8. The method according to claim 6 in which the dehalogenating agent is zinc.

9. The method according to claim 6 in which the dehalogenating agent is iron.

10. The method according to claim 6 additionally comprising subsequently isolating the compound of formula (I) by distillation of the reaction mixture.
